(19) United States
(12) Patent Application Publication
(10) Pub. No.: US 2019/0388670 A1
(43) Pub. Date: Dec. 26, 2019

(54) METHOD OF MANUFACTURING MICRONEEDLE ARRAYS USING A TWO MATERIAL MULTI-LAYER SHEET

(71) Applicant: SABIC GLOBAL TECHNOLOGIES B.V., Bergen op Zoom (NL)

(72) Inventors: Johannes DE BROUWER, Oisterwijk (NL); Herwig JUSTER, Bergen op Zoom (NL); Pieter Jan Antoon JANSSEN, Rijsbergen (NL); Marco NEFS, Bergen op Zoom (NL)

(21) Appl. No.: 16/480,810

(22) PCT Filed: Jan. 30, 2018
(86) PCT No.: PCT/IB2018/050569

§ 371 Date: Jul. 25, 2019

Related U.S. Application Data
(60) Provisional application No. 62/452,588, filed on Jan. 31, 2017.

(51) Int. Cl. A61M 37/00; B29C 55/02; B29C 59/02

(52) U.S. Cl. CPC A61M 37/0015; B29C 55/023; B29C 2059/023; A61M 2037/0053; A61M 2037/0023; B29C 59/026

(57) ABSTRACT

A method of forming a microneedle array having alternating layers of material includes transforming a multilayer sheet by cutting, assembling and stretching steps to form a stretched, stacked multilayer sheet. The stretched, stacked multilayer sheet is cut, assembled and stretched to form a film, the film is heated, and at least a portion of the film is caused to be displaced into a plurality of recesses thereby forming a plurality of protrusions at a surface of the film.

### TECHNICAL FIELD

The application concerns forming microneedle arrays formed via variothermal extrusion and embossing techniques.

### BACKGROUND

Microneedles are attractive for delivery of certain therapeutics. These needles may be particularly desirable as a mode of therapeutic delivery because of the potential to replace syringe-with-needle type of injections with a pain free alternative. Microneedles can be virtually painless because they do not penetrate deep enough to contact nerves and only penetrate the outermost layer of the skin, unlike traditional syringes and hypodermic needles. Additionally, a less shallow penetration may also reduce the risk of infection or injury. Microneedles may also facilitate delivery of a more precise dosage of a therapeutic which enables the use of lower doses in treatments. Other advantages of microneedles for drug delivery include the simplified logistics (absence of required cold chain), ability for patient self-administration (no need for doctor, nurse, reduction of people transport). Beyond therapeutic delivery, drug delivery, microneedles have also been investigated for diagnostic applications. Bodily fluids coming out through the punctured skin can be analyzed for e.g. glucose or insulin.

Microneedles often require a manufacturing process that allows mass production at lowest cost, and as a consequence, shortest possible cycle time. To have proper transcription of mold texture and shape to the molded part, high flow may be necessary, especially having low viscosity at extremely high shear rates. Furthermore, good release from the production mold is important to reduce cycle time to improve the cost efficiency. The needles formed therefrom should exhibit good strength to prevent breaking of the microneedle during usage. While there are a number of benefits to the use of microneedles and considerations with respect to forming them, certain challenges remain in microneedle production. It would be beneficial to prepare microneedles that exhibit a certain aspect ratio for a sharp tip and blade to puncture the skin.

### SUMMARY

Aspects of the present disclosure concern a method of forming a microneedle array comprising: transforming a multilayer sheet by: a) cutting the stretched multilayer sheet to form a first multilayer sheet portion and a second multilayer sheet portion, and b) assembling the first multilayer sheet portion and the second multilayer sheet portion so that the first multilayer sheet portion is placed upon the second multilayer sheet to form a stacked multilayer sheet; c) stretching the stacked multilayer sheet to extend the multilayer sheet in at least one dimension to form a stretched, stacked multilayer sheet, transforming the stretched, stacked multilayer sheet by: a) cutting the stretched, stacked multilayer sheet to form a first stretched, stacked multilayer sheet portion and a second stretched, stacked multilayer sheet portion, and b) assembling the first stretched, stacked multilayer sheet portion and the second stretched, stacked multilayer sheet portion so that the first multilayer sheet portion is placed upon the second multilayer sheet to form a second stacked multilayer sheet; c) stretching the second stacked multilayer sheet to extend the second stacked multilayer sheet in at least one dimension to form a film, heating the film; and causing at least a portion of the film to be displaced into plurality of recesses thereby forming a plurality of protrusions at a surface of the film.

Other aspects concern a microneedle array formed by a method comprising transforming a multilayer sheet by: a) cutting the multilayer sheet to form a first multilayer sheet portion and a second multilayer sheet portion, and b) assembling the first multilayer sheet portion and the second multilayer sheet portion so that the first multilayer sheet portion is placed upon the second multilayer sheet to form a stacked multilayer sheet; c) stretching the stacked multilayer sheet to extend the stacked multilayer sheet in at least one dimension to form a stretched, stacked multilayer, transforming the stretched, stacked multilayer sheet by a) cutting the stretched, stacked multilayer sheet to form a first stretched, stacked multilayer sheet portion and a second stretched, stacked multilayer sheet portion, and b) assembling the first stretched, stacked multilayer sheet portion and the second stretched, stacked multilayer sheet portion so that the first stretched, stacked multilayer sheet portion is placed upon the second stretched, stacked multilayer sheet to form a second stretched, stacked multilayer sheet; and c) stretching the stacked multilayer sheet to extend dimensions of the second stretched, stacked multilayer sheet to form a film, heating the film; and causing at least a portion of the film to be displaced into plurality of recesses thereby forming a plurality of protrusions at a surface of the film.

The present disclosure relates to a system for forming a microneedle array, the system comprising: a multilayer sheet wherein a first layer comprises a first polymer and a second layer comprises a second polymer, wherein the multilayer sheet is transformed by a) stretching the multilayer sheet to extend dimensions of the multilayer sheet, b) cutting the stretched multilayer sheet to form a first multilayer sheet portion and a second multilayer sheet portion, and c) assembling the first multilayer sheet portion and the second multilayer sheet portion so that the first multilayer sheet portion is placed upon the second multilayer sheet to form a stacked multilayer sheet; a support element configured to position the transformed stacked multilayer sheet; a counter pressure element disposed adjacent the support element so that the stacked multilayer sheet is disposed between the support element and the counter pressure element, wherein the counter pressure element comprises a plurality of recesses on a surface of the counter pressure element oriented towards the stacked multilayer sheet; and a heat source configured to heat the stacked multilayer sheet

### BRIEF DESCRIPTION OF THE DRAWINGS

FIG. 1 provides a schematic diagram of a multilayer sheet with protective layers according to an aspect of the present disclosure.

FIG. 2 provides a schematic diagram of methods of transforming a multilayer sheet according to an aspect of the present disclosure.

FIG. 3 provides a depiction of a conventional embossing process.

FIG. 4 provides a depiction of an embossing process of a multilayer sheet according to an aspect of the present disclosure.

### DETAILED DESCRIPTION OF ILLUSTRATIVE EMBODIMENTS

The present disclosure can be understood more readily by reference to the following detailed description of the disclosure and the Examples included therein.

Microneedles can be used to deliver a therapeutic or to draw blood without penetrating tissue as deep a traditional needles. Such microneedles can be used individually or as an array of needles. The needles are typically produced via mass production at a low cost. To efficiently function as a therapeutic delivery mechanism or as a diagnostic tool, microneedles must be sufficiently sharp to penetrate dermal surfaces while still maintaining the benefit of being relatively pain free. Thus, a given microneedle production array is desired to exhibit a certain aspect ratio among the formed microneedles while maintaining their structural integrity and strength during use. Conventional microneedle arrays formed from polycarbonate (PC) show good suitable stiffness in application, but may tend to fail in filling the high aspect ratio of microneedle molds (or the microneedle master structure) during processing via injection molding. This may also result in poor replication of the needle tip. Conventional microneedle arrays formed from semi-crystal materials such polybutylene terephthalate (PBT) show good processing capabilities. Ductile behavior of the PBT array may facilitate puncturing of the skin surface. But, the overall mechanical performance reveals that these PBT needles may not be as strong as PC microneedles. The semi-crystal microneedles of PBT for example may tend to bend more and, while needle sharpness is initially good, over time the needle tip may dull. Furthermore, chemical resistance of the microneedle array may fulfill regulatory critical to quality (CTQ) requirements. There should be minimal or no chemical reaction among the active ingredient of the therapeutic, the carrier/coating, and the material forming the microneedle array during production, sterilization, storage, and/or during the use of the microneedle array. Such interactions may destroy or alter the active ingredient, affect needle properties, or both. In various aspects, the microneedle array formed according to the methods described herein exhibit both the strength and ductility that may be lacking in conventional microneedle arrays.

The combination of PC and PBT in a layered structure helps to obtain the stiffness of PC (especially in the tip area) and incorporates the ductile behavior of PBT to form a hybrid microneedle array. The systems and methods of forming a microneedle array having the desired varying aspect ratio, strength, and mechanical performance sufficient to provide a sharp tip among the microneedles and a sharp blade to properly penetrate or cut the skin.

According to aspects of the present disclosure, a method of forming a microneedle array may comprise transforming a multilayer sheet. The multilayer sheet may be transformed by stretching the multilayer sheet to extend the multilayer sheet in at least one dimension, cutting the stretched multilayer sheet to form a first multilayer sheet portion and a second multilayer sheet portion, and assembling the first multilayer sheet portion and the second multilayer sheet portion so that the first multilayer sheet portion is placed upon the second multilayer sheet to form a stacked multilayer sheet. The stacked multilayer sheet may then be transformed. Transforming the stacked multilayer sheet may comprise a) stretching the stacked multilayer sheet to extend at least one dimension of the stacked multilayer sheet, b) cutting the stretched, stacked multilayer sheet to form a first stacked multilayer sheet portion and a second stacked multilayer sheet portion, and c) assembling the first stacked multilayer sheet portion and the second stacked multilayer sheet portion so that the first stacked multilayer sheet portion is placed upon the second stacked multilayer sheet to form a film. The film may be heated and a portion of which may be displaced into a plurality of recesses of a mold to form a plurality of protrusions at a surface of the film.

A multilayer sheet for formation of the microneedle array described herein may comprise at least two layers. Multilayer sheets of the present disclosure are anticipated to comprise a large number of layers. For example, the disclosed multilayer sheet may comprise up to about 512 layers. These layers may comprise various materials. Each layer may comprise a different type of material. In a given multilayer sheet, the sheet may comprise alternating material layers.

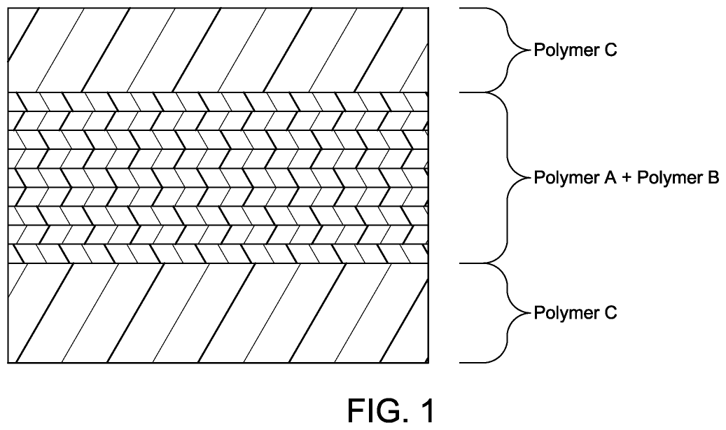

The multilayer sheet may be formed via extrusion of varying materials such as polymers. For example, a multilayer sheet may be formed by the extrusion of alternating polymer materials. FIG. 1 presents an exemplary multilayer sheet showing alternating layers formed from polymer A and polymer B. These alternating polymer layers may include polycarbonate and polybutylene terephthalate (PBT). For example, a first layer may comprise a polycarbonate, a second layer of the multilayer sheet may comprise PBT; a third layer may comprise polycarbonate; and so on and so forth. The multilayer sheet may be formed with protective skin layers. These skin layers, designated polymer C in FIG. 1, may be extruded with the multilayer sheet. That is, a first skin layer may be extruded and a first layer of the multilayer sheet may be extruded thereupon. A second layer of the multilayer sheet may be extruded at the first layer, a third layer may be extruded at the second layer and so on until an n^{th} layer is extruded on a preceding layer. A second skin layer may be extruded at the n^{th} layer of the multilayer sheet. The protective skin layers may prevent physical damage to the multilayer sheet during cooling after extrusion, during storage, or other manipulation. For example, the formed multilayer sheet may be stored as roll before the molding process. Exemplary materials for the skin layer may comprise polypropylene or polycarbonate.

A multilayer sheet of the present disclosure may be transformed. Transforming the multilayer into a stacked multilayer sheet may comprise cutting the multilayer sheet to provide separate portions of the multilayer sheet. The separate portions may then be stacked on atop the other. The cut and stacked multilayer sheet may be stretched. Stretching the multilayer sheet to extends the multilayer sheet in at least one dimension to provide a stretched multilayer sheet. For example, as a sheet is generally planar, the multilayer sheet may be stretched in the x-direction, the y-direction, or both. Stretching of the multilayer sheet may cause a reduction in the thickness of the individual respective layers of the multilayer sheet corresponding to the direction of the stretching. Stretching of the multilayer sheet may cause a reduction of the thickness of the multilayer sheet corresponding to the direction of stretching. Stretching may occur by at least a factor of 1.5 the width of the multilayer sheet. A machine may be used to perform the transformation of the multilayer sheet. An exemplary machine is available from Nordson EDI which includes splitting elements to create layers according to the formula 2ⁿ+1.

The stretched multilayer sheet may then be cut to form a first multilayer sheet portion and a second multilayer sheet portion. The cutting may be performed so that the first multilayer sheet portion and the second multilayer sheet portion are about the same in size. In some examples, the first multilayer sheet portion and the second multilayer sheet portion are not about the same size.

In some examples, the multilayer sheet may be cut before stretching to form a first multilayer sheet portion and a second multilayer sheet portion. That is, cutting may occur before or after stretching of the multilayer sheet in order to provide the first and second multilayer sheet portions. The cutting may be performed so that the first multilayer sheet portion and the second multilayer sheet portion are about the same in size. However, the first multilayer sheet portion and second multilayer sheet portion need not be about the same size.

The first multilayer sheet portion and the second multilayer sheet portion may be assembled. Assembling the first and second multilayer sheet portions may comprise orienting or positioning the sheet portions so that the sheet portions are in contact along a planar surface of each other.

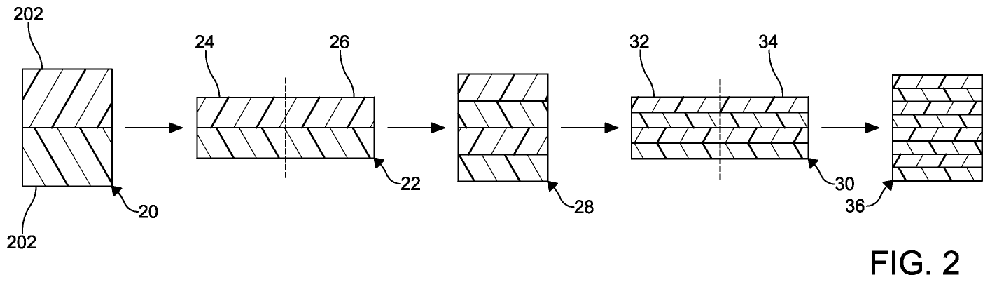

The overall repeated process of stretching, cutting, and stacking, or cutting, stacking, and stretching, may be referred to as a Baker's formation. FIG. 2 shows a Baker's formation of the multilayer sheet of the present disclosure. A multilayer sheet 20 may comprise a first layer 202 and a second layer 204. The multilayer sheet 2 may be stretched laterally, that is in a direction opposite to the direction of layering of the individual layers 202, 204 to provide stretched multilayer sheet 22. Stretched multilayer sheet may then be cut, for example, into two halves to provide a first multilayer sheet portion 24 and a second multilayer sheet portion 26. As shown, each of the first multilayer sheet portion 24 and the second multilayer sheet portion 26 may comprise two layers 202, 204. The first multilayer sheet portion 24 and the second multilayer sheet portion 26 may be stacked adjacent to one another, more specifically, on top of one another, to provide the stacked multilayer sheet 28 thereby completing a first transformation. As shown, stacked multilayer sheet 28 comprises four alternating layers of first layer 202 and second layer 204.

In a second transformation, stacked multilayer sheet 28 may be stretched to provide a second stretched multilayer sheet 30. Second stretched multilayer sheet 30 may be cut, for example, into two halves, to provide a third multilayer sheet portion 32 and a fourth multilayer sheet portion 34. As shown, each of the third multilayer sheet portion 32 and the fourth multilayer sheet portion 34 may comprise four layers, that is, four alternating layers of first layer 202, and second layer 204. The third multilayer sheet portion 32 and the fourth multilayer sheet portion 34 may be stacked adjacent to one another, more specifically, on top of one another, to provide the second stacked multilayer sheet 36 thereby completing a second transformation. The transformations described herein may continue as described until there are 512 alternating layers of first layer 202 and second layer 204, for example.

Alternating layers of the multilayer sheet of the present disclosure may allow for improved chemical resistance. Outer layers of the multilayer sheet, i.e., layers that are in contact with ambient air, may be selected from materials that exhibit chemical resistance or resilience. For example, the material may be resistant to caustic solvents. Moreover, the material may exhibit minimal or no reactivity with active agents of a therapeutic that may be used with the microneedle array.

In some examples, the multilayer sheet may have a width of about 250 micrometers (μm) to about 900 μm prior to molding to form a microneedle array via an appropriate process. Also prior to molding, the skin layer may be removed. Removal of the skin layer may be achieved by physical removal such as peeling.

In certain aspect, the multilayer sheet may be formed, and in some aspects transformed, so that an uppermost layer of the multilayer sheet (or stacked multilayer sheet) comprises polycarbonate. The uppermost layer of the (stacked) multilayer sheet may be situated to be the portion of the sheet that will be contacted with the portion of a microneedle mold that is configured to form the apex or tip of a microneedle in the microneedle array. Polycarbonate disposed at the apex of the microneedle array mold may provide sharp tips for the formed microneedle array. Generally, polycarbonate materials have a higher impact resistance than comparable polyesters such as PBT and thus may maintain a sharper needle tip. PBT as a component of the alternating layers of the multilayer sheet may provide flexibility of the microneedles formed using the array. That is, properties of PBT such as ductility, and other semicrystalline polymers, may allow for formed microneedles to have more durability because the materials allow bending.

Forming a microneedle array may comprise forming a plurality of depressions at a surface of the film comprising the stacked multilayer sheet. Formation of protrusions may occur by heating the film and displacing at least a portion of the film comprising the stacked multilayer sheet into a plurality of recesses corresponding to a configuration for a microneedle array. The plurality of recesses for example may be at least a portion of a mold. The film may be heated up to a minimum of the glass transition temperature of the film and pressure may be applied to displace at least a portion of the film into the plurality of recesses. In a specific example, at least a portion of the film may be displaced into a plurality of recesses of a hot embossing mold.

At least a portion of recesses of the plurality of recesses may exhibit a geometry corresponding to a geometry of a microneedle. For example, a recess may exhibit a half-pyramid geometry where two side lengths of the half-pyramid form an apex, corresponding to a penetrative point of a microneedle formed in the mold. Each recess may thus have a certain base size and apex, as well as an accompanying apex angle. In one example, the plurality of recesses may have a square pyramidal geometry with a base of 100 μm and a side length of 250 μm. In further examples, at least a portion of the plurality of recesses may vary in size relative to each other. This variation in size creates a varying aspect ratio in the microneedle array. For example, side lengths of the half-pyramid geometry of each recess may vary.

In various aspects, the film may be disposed at or adjacent to an embossing mold. Embossing may be used to impart a texture or pattern into a number of products including textiles, paper, synthetic materials, metals, wood, and polymeric materials. In an embossing process, a substrate is caused to conform under pressure to the depths and/or contours of a pattern engraved or otherwise formed on an embossing roll or mold. Embossing may be accomplished by passing a substrate through one or more patterned embossing rolls set to apply a certain pressure and penetration depth to the substrate. As the substrate is maintained within the embossing mold, the pattern on of the mold is imparted onto the substrate.

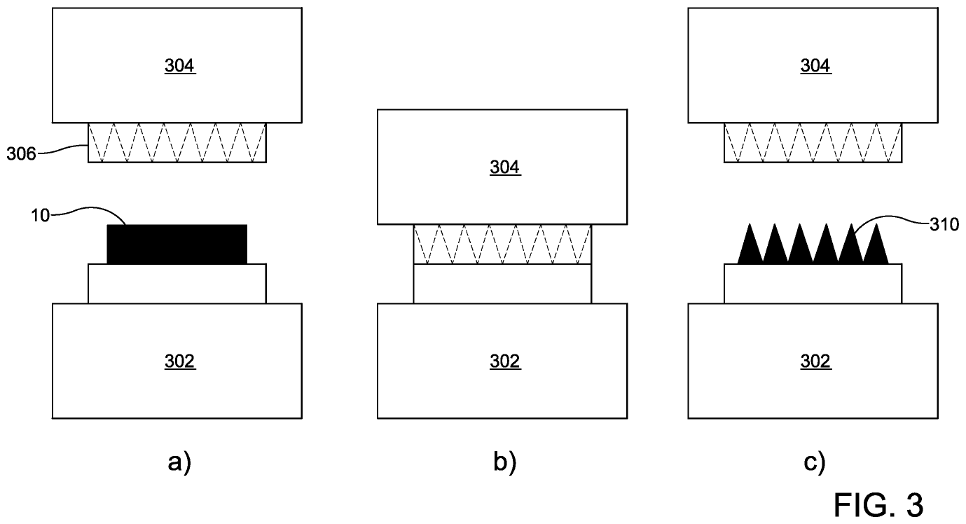

The patterns of a given embossing mold may be mated or non-mated. In a mated embossing mold, the pattern on one portion of the mold may identically, or similarly, compliment, or “mate,” with the pattern on a second or other of the mated rolls. The pattern on a non-mated embossing mold does not match identically with the pattern on the other roll. Depending on the desired results, either type of embossing mold can be used. Various types of embossing processes may be useful in the formation of a microneedle array according to the methods described herein. A conventional hot embossing mold is shown in FIG. 3. Processes of the embossing step are shown as (A), (B), and (C), respectively including placement/heating, molding, and demolding. In a hot embossing process (A), a substrate (in the form of a sheet) 10 may be situated at a lower portion 302 of the hot embossing mold. The substrate may be situated between the lower portion 302 and an upper portion 304 of the hot embossing mold. Heat may be applied to the substrate 10 via the lower portion 302 for example. The upper portion 304 of the hot embossing mold may comprise a texture or pattern 306 to be imparted to the substrate. In molding step (B), the upper and lower portions 304, 302 may be engaged. Engaging the upper and lower portions 304, 302 may refer to contacting the portions 304, 302. Contacting the upper and lower portions 304, 302 causes the substrate to be displaced into the texture or pattern of the upper portion 304 of the hot embossing mold. One or both of the upper and lower portions 304, 302 may be configured to heat the substrate to facilitate displacement of the substrate 10 into the texture or pattern 306 of the upper mold portion 304. In molding step (C), the upper and lower portions 304, 302 may be disengaged to release the mold portions 304, 302 from contact which demolds the substrate 10 to provide a molded part 310 having the texture or pattern 306 of the upper portion 304.

According to various aspects of the present disclosure, hot embossing may be applied to a transformed multilayer sheet as described herein to provide improved production of a microneedle array. The method of the present disclosure may apply hot embossing to a transformed multilayer sheet to provide a microneedle array. A portion of the hot embossing mold may be used as a mold for the microneedle array; the pattern or texture of the hot embossing mold comprising depressions that exhibit an inverse geometry suitable for microneedles.

As described herein, a method of forming a microneedle array may comprise transforming a multilayer sheet by: a) stretching the multilayer sheet to extend the multilayer sheet in at least one dimension, b) cutting the stretched multilayer sheet to form a first multilayer sheet portion and a second multilayer sheet portion, and c) assembling the first multilayer sheet portion and the second multilayer sheet portion so that the first multilayer sheet portion is placed upon the second multilayer sheet to form a stacked multilayer sheet; transforming the stacked multilayer sheet by a) stretching the stacked multilayer sheet to extend at least one dimension of the stacked multilayer sheet, b) cutting the stretched, stacked multilayer sheet to form a first stacked multilayer sheet portion and a second stacked multilayer sheet portion, and c) assembling the first stacked multilayer sheet portion and the second stacked multilayer sheet portion so that the first stacked multilayer sheet portion is placed upon the second stacked multilayer sheet to form a film; heating the film; and causing at least a portion of the film to be displaced into plurality of recesses thereby forming a plurality of protrusions at a surface of the film.

An appropriate system for forming the microneedle array may comprise a multilayer sheet, transformed as described herein, a support element configured to position the transformed stacked multilayer sheet; a counter pressure element disposed adjacent the support element so that the stacked multilayer sheet is disposed between the support element and the counter pressure element, wherein the counter pressure element comprises a plurality of recesses on a surface of the counter pressure element oriented towards the stacked multilayer sheet; and a heat source configured to heat the stacked multilayer sheet. Recesses of the counter pressure element may be formed via machining to achieve recesses having the geometry and sufficient aspect ratios to form a desired microneedle array. As an example, the counter pressure element may comprise a plurality of recesses in an amount of at least about 100 protrusions per square centimeter of a surface of the counter pressure element to be contacted with the stacked multilayer sheet (or film, as described herein).

Machining to form the plurality of recesses of the counter pressure element may be formed via mechanical structuring, via micro electro discharge machining, via laser percussion drilling, or according to LIGA structuring, for example. LIGA may refer to a process combining lithography, electroplating, and replication to form microstructures in steel, for example.

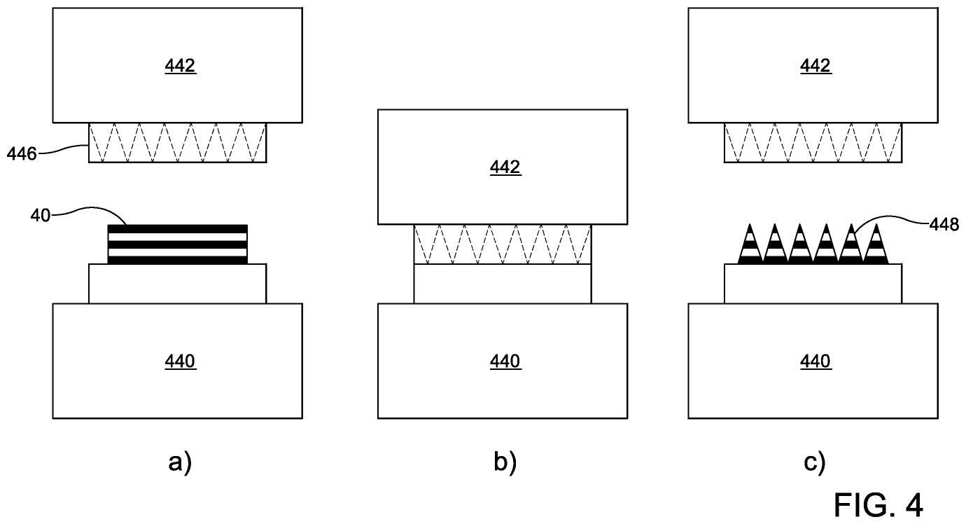

FIG. 4 presents an exemplary hot embossing process according to aspects of the present disclosure. In a hot embossing process (A), a transformed multilayer sheet 40 may be situated at a lower portion 440 of the hot embossing mold. The transformed multilayer sheet 40 may be situated between the lower portion 440 and an upper portion 442 of the hot embossing mold. Heat may be applied to the transformed multilayer sheet 40 via the lower portion 302, for example. The upper portion 442 of the hot embossing mold may comprise a plurality of recesses 446 therein. A recess of the plurality of recesses 446 may correspond to geometry for a microneedle of a microneedle array. Specifically, a recess may have a half pyramid geometry wherein two side lengths of the half pyramid geometry meet to form an apex. The apex of a recess may correspond to a point or tip of a microneedle formed in the hot embossing mold.

In molding step (B), the upper and lower portions 442, 440 may be engaged. Engaging the upper and lower portions 442, 440 may refer to contacting the portions 442, 440. Contacting the upper and lower portions 442, 440 causes at least a portion of multilayer sheet 40 to be displaced into at least a portion of the plurality of recesses 446 of the upper mold portion. The upper and lower portions 442, 440 may further be configured to heat the multilayer sheet 40. In molding step (C), or demolding, the upper and lower portions 442, 440 may be disengaged to release the mold portions 442, 440 from contact to demold to provide a plurality of projections at a surface of the transformed multilayer sheet 40. The upper portion may perform as a counter pressure element to cause the multilayer sheet 40 to be displaced into the plurality of recesses 446. Displacement of the transformed multilayer sheet 40 into the plurality of recesses of the upper portion 442 of the hot embossing mold has thus formed a microneedle array.

In some aspects, the depressions of the upper mold portion may be oriented in a specific repeating pattern. In further examples however, the depressions may be randomly distributed at the upper mold portion. The orientation of depressions in the upper mold portion may thus correspond to a pattern in a resulting microneedle array or may provide a microneedle array in a random configuration.

As provided herein, the (transformed) multilayer sheet may be oriented in the hot embossing mold so that so that an uppermost layer of the multilayer sheet (or stacked multilayer sheet) comprises polycarbonate. The uppermost layer of the (transformed) multilayer sheet may refer to a layer of the sheet that is closest to a portion of the mold comprising a pattern or texture for replication. Here, the mold comprising a pattern or texture for replication may be a microneedle mold or microneedle microstructure mold. Thus, the uppermost layer of the multilayer sheet may be oriented in the hot embossing molding apparatus so that the uppermost layer that will be contacted with the portion of a microneedle mold that is configured to form the apex or tip of a microneedle in the microneedle array. In some examples, polycarbonate may be desirable as the uppermost layer of the multilayer sheet that is to be disposed at the apex of the microneedle array mold may provide sharp tips for the formed microneedle array. Depending on which portion of a given microstructure mold has the microneedle microstructures therein; the multilayer sheet may be oriented so that a layer comprising polycarbonate is oriented towards the portion of the mold comprising the microstructures.

Given the alternating layers of the multilayer sheet described herein, microneedles formed according to the present disclosure may have the benefit of a strong tip, such as PC, and a durable microneedle body. Certain materials may be used to provide durability and some flexibility to the body of microneedles formed from the multilayer sheet described herein. These materials may include polyesters as semicrystalline polymers that provide ductility. Other exemplary polyesters may include polyethylene terephthalate (PET), polyethylene terephthalate glycol-modified (PETG), glycol-modified poly-cyclohexylenedimethylene terephthalate (PCTG), polycyclohexylenedimethylene terephthalate (PCT), isophthalic acid-modified polycyclohexylenedimethylene terephthalate (PCTA), and Tritan™ (a combination of dimethyl terephthalate, 1,4-cyclohexanedimethanol, and 2,2,4,4-tetramethyl-1,3-cyclobutanediol from Eastman Chemical). PBT as a component of the alternating layers of the multilayer sheet may provide flexibility of the body of microneedles formed using the systems and methods described herein. A specific PBT resin may include a resin from the Valox™ line of PBT and/or PET resins available from SABIC™. Xylex™, a combination of PC and an amorphous polyester, is another useful resin.

In various aspects, the multilayer sheet may comprise alternating layers of polymeric material. The multilayer sheet for forming a microneedle array in the disclosed hot embossing process may be heated within the mold to a temperature at least about the minimum glass transition temperature of the alternating polymeric materials.

The multilayer sheet may comprise alternating layers of polycarbonate. The terms “polycarbonate” or “polycarbonates” as used herein includes copolycarbonates, homopolycarbonates and (co)polyester carbonates. The term polycarbonate can be further defined as compositions have repeating structural units of the formula (1):

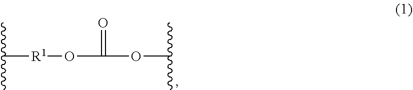

in which at least 60 percent of the total number of R1 groups are aromatic organic radicals and the balance thereof are aliphatic, alicyclic, or aromatic radicals. In a further aspect, each R1 is an aromatic organic radical and, more preferably, a radical of the formula (2):

-A1-Y1-A2- (2),

wherein each of A1 and A2 is a monocyclic divalent aryl radical and Y1 is a bridging radical having one or two atoms that separate A1 from A2. In various aspects, one atom separates A1 from A2. For example, radicals of this type include, but are not limited to, radicals such as —O—, —S—, —S(O)—, —S(O2)—, —C(O)—, methylene, cyclohexyl-methylene, 2-[2.2.1]-bicycloheptylidene, ethylidene, isopropylidene, neopentylidene, cyclohexylidene, cyclopentadecylidene, cyclododecylidene, and adamantylidene. The bridging radical Y1 is preferably a hydrocarbon group or a saturated hydrocarbon group such as methylene, cyclohexylidene, or isopropylidene. Polycarbonate materials include materials disclosed and described in U.S. Pat. No. 7,786,246, the disclosure of which is incorporated herein by this reference in its entirety. Polycarbonate polymers can be manufactured by means known to those skilled in the art. An exemplary polycarbonate may include LEXAN™ HF1110.

Polybutylene terephthalate (PBT) refers to a semicrystalline thermoplastic material. PBT is often formed via the polymerization of butanediol and a purified terephthalic acid.

An exemplary polymer of the present disclosure may include additives such as a mold release agent to facilitate ejection of a formed microneedle array from the mold assembly. Examples of mold release agents include both aliphatic and aromatic carboxylic acids and their alkyl esters, for example, stearic acid, behenic acid, pentaerythritol tetrastearate, glycerin tristearate, and ethylene glycol distearate. Polyolefins such as high-density polyethylene, linear low-density polyethylene, low-density polyethylene, and similar polyolefin homopolymers and copolymers can also be used a mold release agents. Some compositions use pentaerythritol tetrastearate, glycerol monostearate, a wax or a poly alpha olefin. Mold release agents are typically present in the composition at 0.05 to 10 wt %, based on total weight of the composition, specifically 0.1 to 5 wt %, 0.1 to 1 wt % or 0.1 to 0.5 wt %. Some preferred mold release agents will have high molecular weight, typically greater than 300, to prevent loss of the release agent from the molten polymer mixture during melt processing.

The polymer material for forming the microneedle array may further comprise one or more additives intended to impart certain characteristics to a microneedle array formed by the mold assembly described herein. The polymer material may include one or more of an impact modifier, flow modifier, antioxidant, heat stabilizer, light stabilizer, ultraviolet (UV) light stabilizer, UV absorbing additive, plasticizer, lubricant, antistatic agent, antimicrobial agent, colorant (e.g., a dye or pigment), surface effect additive, radiation stabilizer, or a combination comprising one or more of the foregoing. For example, a combination of a heat stabilizer, and ultraviolet light stabilizer can be used. In general, the additives are used in the amounts generally known to be effective. For example, the total amount of the additive composition can be 0.001 to 10.0 wt %, or 0.01 to 5 wt %, each based on the total weight of all ingredients in the composition.

The polymer material may include various additives ordinarily incorporated into polymer compositions, with the proviso that the additive(s) are selected so as to not significantly adversely affect the desired properties of the material (good compatibility for example). Such additives can be mixed at a suitable time during the mixing of the components for forming the material comprising the multilayer sheet. In addition, the polymer material may exhibit excellent release, as measured by ejection force (N) and coefficient of friction. The polymer material also preferably show (i) high flow at high shear conditions to allow good transcription of mold texture and excellent filling of the finest mold features, (ii) good strength and impact (as indicated by ductile Izod Notched Impact at room temperature and modulus), and (iii) high release to have efficient de-molding and reduced cooling and cycle time during molding. The microneedles formed herein may have sufficient mechanical strength to remain intact (i) while being inserted into the biological barrier, (ii) while remaining in place for up to a number of days, and (iii) while being removed.

### Definitions

It is to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural equivalents unless the context clearly dictates otherwise. Thus, for example, reference to “a polycarbonate polymer” includes mixtures of two or more polycarbonate polymers.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±5% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein the terms “weight percent,” “weight %,” and “wt. %” of a component, which can be used interchangeably, unless specifically stated to the contrary, are based on the total weight of the formulation or composition in which the component is included. For example if a particular element or component in a composition or article is said to have 8% by weight, it is understood that this percentage is relative to a total compositional percentage of 100% by weight.

As used herein, the terms “weight average molecular weight” or “Mw” can be used interchangeably, and are defined by the formula:

$${M}_{w} = \frac{\sum {N}_{i} {M}_{i}^{2}}{\sum {N}_{i} {M}_{i}} ,$$

where Mi is the molecular weight of a chain and Ni is the number of chains of that molecular weight. Mw can be determined for polymers, e.g. polycarbonate polymers, by methods well known to a person having ordinary skill in the art using molecular weight standards, e.g. polycarbonate standards or polystyrene standards, preferably certified or traceable molecular weight standards. Polystyrene basis refers to measurements using a polystyrene standard.

The term “siloxane” refers to a segment having a Si—O—Si linkage.

The term “flowable” means capable of flowing or being flowed. Typically a polymer is heated such that it is in a melted state to become flowable.

### ASPECTS

The present disclosure comprises at least the following aspects.

Aspect 1. A method of forming a microneedle array comprising: transforming a multilayer sheet by: a) cutting the stretched multilayer sheet to form a first multilayer sheet portion and a second multilayer sheet portion, and b) assembling the first multilayer sheet portion and the second multilayer sheet portion so that the first multilayer sheet portion is placed upon the second multilayer sheet to form a stacked multilayer sheet; c) stretching the stacked multilayer sheet to extend the multilayer sheet in at least one dimension to form a stretched, stacked multilayer sheet, transforming the stretched, stacked multilayer sheet by: a) cutting the stretched, stacked multilayer sheet to form a first stretched, stacked multilayer sheet portion and a second stretched, stacked multilayer sheet portion, and b) assembling the first stretched, stacked multilayer sheet portion and the second stretched, stacked multilayer sheet portion so that the first multilayer sheet portion is placed upon the second multilayer sheet to form a second stacked multilayer sheet; c) stretching the second stacked multilayer sheet to extend the second stacked multilayer sheet in at least one dimension to form a film, heating the film; and causing at least a portion of the film to be displaced into plurality of recesses thereby forming a plurality of protrusions at a surface of the film.

Aspect 2. A method of forming a microneedle array comprising: transforming a multilayer sheet by: a) stretching the multilayer sheet to extend the multilayer sheet in at least one dimension, b) cutting the stretched multilayer sheet to form a first multilayer sheet portion and a second multilayer sheet portion, and c) assembling the first multilayer sheet portion and the second multilayer sheet portion so that the first multilayer sheet portion is placed upon the second multilayer sheet to form a stacked multilayer sheet; transforming the stacked multilayer sheet by a) stretching the stacked multilayer sheet to extend at least one dimension of the stacked multilayer sheet, b) cutting the stretched, stacked multilayer sheet to form a first stacked multilayer sheet portion and a second stacked multilayer sheet portion, and c) assembling the first stacked multilayer sheet portion and the second stacked multilayer sheet portion so that the first stacked multilayer sheet portion is placed upon the second stacked multilayer sheet to form a film; heating the film; and causing at least a portion of the film to be displaced into plurality of recesses thereby forming a plurality of protrusions at a surface of the film.

Aspect 3. The method of any one of aspects 1-2, wherein the plurality of protrusions correspond to a configuration for a microneedle array.

Aspect 4. The method of any one of aspects 1-3, further comprising repeating transforming of the film to provide up to about 512 stacked, stretched layers prior to heating the film.

Aspect 5. The method of any one of aspects 1-4, wherein each layer of the multilayer sheet comprises a material that has a glass transition temperature and wherein heating the film comprises heating the film to a temperature lower than a lowest glass transition temperature of the layers of the multilayer sheet.

Aspect 6. The method of any one of aspects 1-5, wherein the multilayer sheet comprises at least a first layer and a second layer, wherein the first layer comprises a first polymer and the second layer comprises a second polymer.

Aspect 7. The method of aspect 6, wherein the first layer comprises a polycarbonate.

Aspect 8. The method of aspect 6, wherein the first layer comprises LEXAN™ HF1110.

Aspect 9. The method of aspect 6, wherein the first layer comprises a polycarbonate and second layer comprises a polyester.

Aspect 10. The method of aspect 9, wherein the polyester comprises a PBT, PET, PCT, PCTG, or any combination thereof.

Aspect 11. The method of aspect 6, wherein the first layer comprises a polycarbonate and the second layer comprises polybutylene terephthalate.

Aspect 12. The method of any one of aspects 1-11, wherein the causing the film to be formed into a plurality of recesses comprises advancing the film between a support element and a counter pressure element, wherein the counter pressure element comprises the plurality of recesses, and wherein advancing the film displaces at least a portion of the film.

Aspect 13. The method of aspect 12, wherein the counter pressure element comprises a plurality of recesses in an amount of at least about 100 protrusions per square centimeter of a surface of the counter pressure element in contact with the film.

Aspect 14. The method of any one of aspects 12-13, wherein the counter pressure element comprises steel.

Aspect 15. The method of any one of aspects 12-14, wherein the recesses of the counter pressure element are formed via LIGA structuring.

Aspect 16. The method of any one of aspects 12-15, wherein the plurality of recesses of the counter pressure element is formed via mechanical structuring.

Aspect 17. The method of any one of aspects 12-16, wherein the plurality of recesses of the counter pressure element is formed via micro electro discharge machining or laser percussion drilling.

Aspect 18. The method of any one of aspects 1-17, wherein the multilayer sheet is formed via extrusion of a first layer comprising a first polymer and extrusion of a second layer comprising a second polymer onto the first layer comprising the first polymer.

Aspect 19. The method of any one of aspects 1-18, wherein a first skin layer is applied to a first surface of the film and a second skin layer is applied to a second surface of the film.

Aspect 20. The method of any one of aspects 1-19, wherein the first skin layer and the second skin layer comprises polypropylene.

Aspect 21. The method of any one of aspects 1-20, wherein the plurality of recesses exhibits half pyramidal geometries.

Aspect 22. The method of any one of aspects 1-21, wherein a recess of the plurality of recesses has a square pyramidal geometry with a base of 100 micrometers by 100 micrometers and a side length of 250 micrometers.

Aspect 23. The method of any one of aspects 1-22, wherein the plurality of recesses corresponds to a configuration for a microneedle array.

Aspect 24. A microneedle array formed by a method comprising: transforming a multilayer sheet by: a) cutting the multilayer sheet to form a first multilayer sheet portion and a second multilayer sheet portion, and b) assembling the first multilayer sheet portion and the second multilayer sheet portion so that the first multilayer sheet portion is placed upon the second multilayer sheet to form a stacked multilayer sheet; c) stretching the stacked multilayer sheet to extend the stacked multilayer sheet in at least one dimension to form a stretched, stacked multilayer, transforming the stretched, stacked multilayer sheet by a) cutting the stretched, stacked multilayer sheet to form a first stretched, stacked multilayer sheet portion and a second stretched, stacked multilayer sheet portion, and b) assembling the first stretched, stacked multilayer sheet portion and the second stretched, stacked multilayer sheet portion so that the first stretched, stacked multilayer sheet portion is placed upon the second stretched, stacked multilayer sheet to form a second stretched, stacked multilayer sheet; and c) stretching the stacked multilayer sheet to extend dimensions of the second stretched, stacked multilayer sheet to form a film, heating the film; and causing at least a portion of the film to be displaced into plurality of recesses thereby forming a plurality of protrusions at a surface of the film.

Aspect 25. A microneedle array formed by a method comprising: transforming a multilayer sheet by: a) stretching the multilayer sheet to extend the multilayer sheet in at least one dimension, b) cutting the stretched multilayer sheet to form a first multilayer sheet portion and a second multilayer sheet portion, and c) assembling the first multilayer sheet portion and the second multilayer sheet portion so that the first multilayer sheet portion is placed upon the second multilayer sheet to form a stacked multilayer sheet; transforming the stacked multilayer sheet by a) stretching the stacked multilayer sheet to extend dimensions of the stacked multilayer sheet, b) cutting the stretched, stacked multilayer sheet to form a first stacked multilayer sheet portion and a second stacked multilayer sheet portion, and c) assembling the first stacked multilayer sheet portion and the second stacked multilayer sheet portion so that the first stacked multilayer sheet portion is placed upon the second stacked multilayer sheet to form a film; heating the film; and causing at least a portion of the film to be displaced into plurality of recesses thereby forming a plurality of protrusions at a surface of the film.

Aspect 26. A system for forming a microneedle array, the system comprising: a multilayer sheet wherein a first layer comprises a first polymer and a second layer comprises a second polymer, wherein the multilayer sheet is transformed by a) cutting the multilayer sheet to form a first multilayer sheet portion and a second multilayer sheet portion, b) assembling the first multilayer sheet portion and the second multilayer sheet portion so that the first multilayer sheet portion is placed upon the second multilayer sheet to form a stacked multilayer sheet, and c) stretching the stacked multilayer sheet to extend dimensions of the multilayer sheet to form a stretched, stacked multilayer sheet; and a support element configured to position the transformed stacked multilayer sheet; a counter pressure element disposed adjacent the support element so that the stacked multilayer sheet is disposed between the support element and the counter pressure element, wherein the counter pressure element comprises a plurality of recesses on a surface of the counter pressure element oriented towards the stretched, stacked multilayer sheet; and a heat source configured to heat the stretched, stacked multilayer sheet.

Aspect 27. A system for forming a microneedle array, the system comprising: a multilayer sheet wherein a first layer comprises a first polymer and a second layer comprises a second polymer, wherein the multilayer sheet is transformed by a) stretching the multilayer sheet to extend dimensions of the multilayer sheet, b) cutting the stretched multilayer sheet to form a first multilayer sheet portion and a second multilayer sheet portion, and c) assembling the first multilayer sheet portion and the second multilayer sheet portion so that the first multilayer sheet portion is placed upon the second multilayer sheet to form a stacked multilayer sheet; a support element configured to position the transformed stacked multilayer sheet; a counter pressure element disposed adjacent the support element so that the stacked multilayer sheet is disposed between the support element and the counter pressure element, wherein the counter pressure element comprises a plurality of recesses on a surface of the counter pressure element oriented towards the stacked multilayer sheet; and a heat source configured to heat the stacked multilayer sheet.

Aspect 28. The system of aspect 27, wherein the multilayer sheet is transformed to form a stretched, stacked multilayer sheet having up to about 512 layers.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method of forming a microneedle array comprising:
transforming a multilayer sheet by
a) cutting the stretched multilayer sheet to form a first multilayer sheet portion and a second multilayer sheet portion,
b) assembling the first multilayer sheet portion and the second multilayer sheet portion so that the first multilayer sheet portion is placed upon the second multilayer sheet portion to form a stacked multilayer sheet,
c) stretching the stacked multilayer sheet to extend the multilayer sheet in at least one dimension to form a stretched, stacked multilayer sheet;
transforming the stretched, stacked multilayer sheet by
a) cutting the stretched, stacked multilayer sheet to form a first stretched, stacked multilayer sheet portion and a second stretched, stacked multilayer sheet portion,
b) assembling the first stretched, stacked multilayer sheet portion and the second stretched, stacked multilayer sheet portion so that the first multilayer sheet portion is placed upon the second multilayer sheet portion to form a second stacked multilayer sheet, and
c) stretching the second stacked multilayer sheet to extend the second stacked multilayer sheet in at least one dimension to form a film;
heating the film; and
causing at least a portion of the film to be displaced into a plurality of recesses thereby forming a plurality of protrusions at a surface of the film.

2. The method of claim 1, wherein the plurality of protrusions correspond to a configuration for a microneedle array.

3. The method of claim 1, further comprising repeating transforming of the film to provide up to about 512 layers.

4. The method of claim 1, wherein each layer of the multilayer sheet comprises a material that has a glass transition temperature and wherein heating the film comprises heating the film to a temperature lower than a lowest glass transition temperature of the layers of the multilayer sheet.

5. The method of claim 1, wherein the multilayer sheet comprises at least a first layer and a second layer, wherein the first layer comprises a first polymer and the second layer comprises a second polymer.

6. The method of claim 5, wherein the first layer comprises a polycarbonate and the second layer comprises polybutylene terephthalate.

7. The method of claim 1, wherein the causing the film to be formed into a plurality of recesses comprises advancing the film between a support element and a counter pressure element, wherein the counter pressure element comprises the plurality of recesses, and wherein advancing the film displaces at least a portion of the film.

8. The method of claim 7, wherein the counter pressure element comprises a plurality of recesses in an amount of at least about 100 protrusions per square centimeter of a surface of the counter pressure element in contact with the film.

9. The method of claim 7, wherein the counter pressure element comprises steel.

10. The method of claim 7, wherein the recesses of the counter pressure element are formed via LIGA structuring.

11. The method of claim 7, wherein the plurality of recesses of the counter pressure element is formed via mechanical structuring.

12. The method of claim 7, wherein the plurality of recesses of the counter pressure element is formed via micro electro discharge machining or laser percussion drilling.

13. The method of claim 1, wherein the multilayer sheet is formed via extrusion of a first layer comprising a first polymer and extrusion of a second layer comprising a second polymer onto the first layer comprising the first polymer.

14. The method of claim 1, wherein a first skin layer is applied to a first surface of the film and a second skin layer is applied to a second surface of the film.

15. The method of claim 1, wherein the plurality of recesses exhibits half pyramidal geometries.

16. The method of claim 1, wherein a recess of the plurality of recesses has a square pyramidal geometry with a base of 100 micrometers by 100 micrometers and a side length of 250 micrometers.

17. The method of claim 1, wherein the plurality of recesses corresponds to a configuration for a microneedle array.

18-20. (canceled)
